SEQUENCE LISTING (69 sequences)

SEQ ID NO: 1 - protein, 107 aa, Artificial
  light chain, variable region of LC.001, LC.059 and LC.063
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 2 - protein, 120 aa, Artificial
  heavy chain, variable region of LC.001
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 3 - protein, 107 aa, Artificial
  light chain, variable region of LC.005
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSFTFGPGTKVDIK

SEQ ID NO: 4 - protein, 120 aa, Artificial
  heavy chain, variable region of LC.005
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSS

SEQ ID NO: 5 - protein, 107 aa, Artificial
  light chain, variable region of LC.010
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFTFGPGTKVDIK

SEQ ID NO: 6 - protein, 120 aa, Artificial
  heavy chain, variable region of LC.010
QVQLVESGGGVVQPGRSLRLSCAASGFTFSNFGMHWVRQAPGKGLEWVAAIWYDGHDKYYAYYVKGRFTISRDNSKNTLFLQMNSLRAEDTAVYYCARDSSSWYRYFDYWGQGTLVTVSS

SEQ ID NO: 7 - protein, 58 aa, Artificial
  light chain, variable region of LC.029
MLHPLCKVGSHQGSVAVDLGQISLSPSAACSLKILQLITVNSIIVSLTFGGGTKVEIK

SEQ ID NO: 8 - protein, 120 aa, Artificial; identical to SEQ ID NO: 4
  heavy chain, variable region of LC.029

SEQ ID NO: 9 - protein, 57 aa, Artificial
  light chain, variable region of LC.019
MPPVWKVGSHQGSAAVDLGQISLSPSAACSLKILQLITVNSLIVTLTFGGGTKVEIK

SEQ ID NO: 10 - protein, 116 aa, Artificial
  heavy chain, variable region of LC.019
QVQLVESGGGVVQPGRSLRLSCAASGFTFSSYTMHWVRQAPGKGLEWVAVIWYDGSNKYYVDSVKGRFTISRDNSKNTLYLQMNSLRAEDTAVYYCARKNWSFDFWGQGTLVTVSS

SEQ ID NO: 11 - protein, 106 aa, Artificial
  light chain, variable region of LC.033 (a)
EIVLTQSPATLSLSPGERATLSCRASQGVSRYLAWYQQKPGQAPRLLIYDASNRATGIPARVSGSGPGTDFTLTISSLEPEDFAVDYCQQRSNWQYTFGQGTKLEI

SEQ ID NO: 12 - protein, 121 aa, Artificial
  heavy chain, variable region of LC.033
QKQLVEFGGGVVQPGRSLRLSCAASGFTFSNYGMHWVRQAPGKGLEWVAVIWNDGSNKYYVDSVKGRFIISRDNSKNTLYLQMNSLRAEDTAVYYCARDRMGIYYYGMDVWGQGTTVTVSS

SEQ ID NO: 13 - protein, 107 aa, Artificial [sequence omitted]

SEQ ID NO: 14 - protein, 330 aa, Artificial [sequence omitted]

SEQ ID NO: 15 - protein, 327 aa, Artificial
  heavy chain,  constant region (gamma 4)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 104 aa, Artificial
  light chain, variable region of LC.033 (b)
EIVLTQSPATLSLSPGERATLSCRASQSVSSYLAWYQQKPGQAPRLLIYDASNRATGIPARFSGSGSGTDFTLTISSLEPEDFAVYYCQQRSNWTFGQGTKVEI

SEQ ID NO: 17 - protein, 120 aa, Artificial
  heavy chain, variable region of LC.059
EVQLLESGGGLVQPGGSLRLSCAASGFTFSSYAMNWVRQAPGKGLEWVSIISGSGGFTYYADSVKGRFTISRDNSKNTLYLQMNRLRAEDTAIYFCAKDDIPAAGTFDPWGQGTLVTVSS

SEQ ID NO: 18 - protein, 106 aa, Artificial
  light chain, variable region of LC.060
AIQLTQSPSSLSASVGDRVTITCRASQGISSALAWYQQKPGKAPKLLIYDVSSLESGVPSRFSGSGSGTDFTLTISSLQPEDFATYYCQQFNSYWTFGQGTKVEIK

SEQ ID NO: 19 - protein, 119 aa, Artificial
  heavy chain, variable region of LC.060
EVQLLESGGGLVQPGGSLRLSCAASGFTFSSYAMSWVRQAPGKGLEWVSLISGSGGLTKYADSVKGRFTISRDNSKRTLYLQMNSLRAEDTAVYYCAKDILVTGALDYWGQGTLVTVSS

SEQ ID NO: 20 - protein, 120 aa, Artificial
  heavy chain, variable region of LC.063
EVQLLESGGGLVQPGGSLRLSCAASGFTFSSYAMNWVRQAPGKGLEWVSIISGSGGFTYYADSVKGRFTISRDNSKKTLYLQMSRLRAEDTAIYFCAKDDIPAAGTFDPWGQGTLVTVSS

SEQ ID NO: 21 - protein, 5 aa, Artificial
  CDR / antibody fragment
SYTMH

SEQ ID NO: 22 - protein, 5 aa, Artificial
  CDR / antibody fragment
SYAMS

SEQ ID NO: 23 - protein, 5 aa, Artificial
  CDR / antibody fragment
NFGMH

SEQ ID NO: 24 - protein, 5 aa, Artificial
  CDR / antibody fragment
NYGMH

SEQ ID NO: 25 - protein, 5 aa, Artificial
  CDR / antibody fragment
SYAMN

SEQ ID NO: 26 - protein, 17 aa, Artificial
  CDR / antibody fragment
IISGSGGFTYYADSVKG

SEQ ID NO: 27 - protein, 17 aa, Artificial
  CDR / antibody fragment
AIWYDGHDKYYSYYVKG

SEQ ID NO: 28 - protein, 17 aa, Artificial
  CDR / antibody fragment
AIWYDGHDKYYAYYVKG

SEQ ID NO: 29 - protein, 17 aa, Artificial
  CDR / antibody fragment
VIWYDGSNKYYVDSVKG

SEQ ID NO: 30 - protein, 17 aa, Artificial
  CDR / antibody fragment
VIWNDGSNKYYVDSVKG

SEQ ID NO: 31 - protein, 18 aa, Artificial
  CDR / antibody fragment
IISGSGGFTYYADSVKGR

SEQ ID NO: 32 - protein, 18 aa, Artificial
  CDR / antibody fragment
LISGSGGLTKYADSVKGR

SEQ ID NO: 33 - protein, 11 aa, Artificial
  CDR / antibody fragment
DSSSWYRYFDY

SEQ ID NO: 34 - protein, 11 aa, Artificial
  CDR / antibody fragment
DRLVAPGTFDY

SEQ ID NO: 35 - protein, 7 aa, Artificial
  CDR / antibody fragment
KNWSFDF

SEQ ID NO: 36 - protein, 12 aa, Artificial
  CDR / antibody fragment
DRMGIYYYGMDV

SEQ ID NO: 37 - protein, 12 aa, Artificial
  CDR / antibody fragment
KDDIPAAGTFDP

SEQ ID NO: 38 - protein, 11 aa, Artificial
  CDR / antibody fragment
KDILVTGALDY

SEQ ID NO: 39 - protein, 11 aa, Artificial
  CDR / antibody fragment
RASQGISSWLA

SEQ ID NO: 40 - protein, 12 aa, Artificial
  CDR / antibody fragment
RASQSVSSSYLA

SEQ ID NO: 41 - protein, 12 aa, Artificial
  CDR / antibody fragment
RASQSVSSNYLA

SEQ ID NO: 42 - protein, 11 aa, Artificial
  CDR / antibody fragment
RASQGVSRYLA

SEQ ID NO: 43 - protein, 11 aa, Artificial
  CDR / antibody fragment
RASQSVSSYLA

SEQ ID NO: 44 - protein, 24 aa, Artificial
  CDR / antibody fragment
LSASVGDRVTITCRASQGISSALA

SEQ ID NO: 45 - protein, 7 aa, Artificial
  CDR / antibody fragment
GASSRAT

SEQ ID NO: 46 - protein, 7 aa, Artificial
  CDR / antibody fragment
AASSLQS

SEQ ID NO: 47 - protein, 7 aa, Artificial
  CDR / antibody fragment
MPPVWKV

SEQ ID NO: 48 - protein, 7 aa, Artificial
  CDR / antibody fragment
DASNRAT

SEQ ID NO: 49 - protein, 7 aa, Artificial
  CDR / antibody fragment
LHPLCKV

SEQ ID NO: 50 - protein, 7 aa, Artificial
  CDR / antibody fragment
DVSSLES

SEQ ID NO: 51 - protein, 8 aa, Artificial
  CDR / antibody fragment
NSLIVTLT

SEQ ID NO: 52 - protein, 9 aa, Artificial
  CDR / antibody fragment
QQYNSYPYT

SEQ ID NO: 53 - protein, 8 aa, Artificial
  CDR / antibody fragment
QQYGSSFT

SEQ ID NO: 54 - protein, 9 aa, Artificial
  CDR / antibody fragment
QQRSNWQYT

SEQ ID NO: 55 - protein, 7 aa, Artificial
  CDR / antibody fragment
QQRSNWT

SEQ ID NO: 56 - protein, 8 aa, Artificial
  CDR / antibody fragment
NSIIVSLT

SEQ ID NO: 57 - protein, 9 aa, Artificial
  CDR / antibody fragment
QQFNSYWTF

SEQ ID NO: 58 - protein, 450 aa, Artificial
  heavy chain of LC.001 (human IgG1 type)
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 450 aa, Artificial
  heavy chain of LC.001 (L234A, L235A human IgG1 mutant)
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 447 aa, Artificial
  heavy chain of LC.001 (S228P human IgG4 mutant)
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 214 aa, Artificial
  light chain of LC.001
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 450 aa, Artificial
  heavy chain of LC.005 (human IgG1 type)
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 450 aa, Artificial
  heavy chain of LC.005 (L234A, L235A human IgG1 mutant)
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 447 aa, Artificial
  heavy chain of LC.005 (S228P human IgG4 mutant)
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 214 aa, Artificial
  light chain of LC.005
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 449 aa, Artificial
  heavy chain of LC.060 (human IgG1 type)
EVQLLESGGGLVQPGGSLRLSCAASGFTFSSYAMSWVRQAPGKGLEWVSLISGSGGLTKYADSVKGRFTISRDNSKRTLYLQMNSLRAEDTAVYYCAKDILVTGALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 449 aa, Artificial
  heavy chain LC.060 (L234A, L235A human IgG1 mutant)
EVQLLESGGGLVQPGGSLRLSCAASGFTFSSYAMSWVRQAPGKGLEWVSLISGSGGLTKYADSVKGRFTISRDNSKRTLYLQMNSLRAEDTAVYYCAKDILVTGALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 446 aa, Artificial
  heavy chain of LC.060 (S228P human IgG4 mutant)
EVQLLESGGGLVQPGGSLRLSCAASGFTFSSYAMSWVRQAPGKGLEWVSLISGSGGLTKYADSVKGRFTISRDNSKRTLYLQMNSLRAEDTAVYYCAKDILVTGALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 213 aa, Artificial
  light chain of LC.060
AIQLTQSPSSLSASVGDRVTITCRASQGISSALAWYQQKPGKAPKLLIYDVSSLESGVPSRFSGSGSGTDFTLTISSLQPEDFATYYCQQFNSYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2010/0098712 A1
(43) Pub. Date: Apr. 22, 2010

(54) Pharmaceutical formulation of an antibody against OX40L

(76) Inventors: Michael Adler, Freiburg (DE); Hanns-Christian Mahler, Basel (CH); Christine Wurth, Loerrach (DE)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 12/454,598

(22) Filed: May 20, 2009

(30) Foreign Application Priority Data
May 20, 2008 (EP) 08156579.8

(51) Int. Cl. A61K 39/395; A61P 37/08

(52) U.S. Cl. 424/172.1

(57) ABSTRACT

Pharmaceutical formulations of an antibody against OX40L and processes for making the same.

### CROSS REFERENCE TO PRIOR APPLICATIONS

This application claims the benefit of priority under 35 USC §119 to European Application No. EP 08156579.8, filed on May 20, 2008, the contents of which are hereby incorporated in their entirety by reference.

### FIELD OF THE INVENTION

This invention relates to pharmaceutical formulations of an antibody against OX40 ligand (OX40L), and processes for the preparation and uses of the formulations.

### BACKGROUND OF THE INVENTION

Human OX40L (gp34, SwissProt P23510) is expressed on activated B cells and dendritic cells upon CD40/CD40L ligation, and on endothelial cells in inflammatory tissues (Review: Weinberg, A. D., Trends Immunol. 23 (2002) 102-109). It has first been isolated from HTLV-1 infected human leukemic cells (immortalization of these T-cells by generation of an autokrine loop with OX40). OX40L and antibodies against are mentioned e.g. in WO 95/12673; WO 95/21915; WO 99/15200; Baum, P. R., et al., EMBO J. 13 (1994) 3992-4001; Imura, A., et al., Blood 89 (1997) 2951-2958; Imura, A., et al., J. Exp. Med. 183 (1996) 2185-2195; Kjaergaard, J., et al., J. Immunol. 167 (2001) 6669-6677; Lane, P., J. Exp. Med. 191 (2000) 201-206; Mallett, S., and Barclay, A. N., Immunol. Today 12 (1991) 220-223; Mallett, S., et al., EMBO J. 9 (1990) 1063-1068; Ndhlovu, L. C., et al., J. Immunol. 167 (2001) 2991-2999; Ohshima, Y., et al., J. Immunol. 159 (1997) 3838-3848; Rogers, P. R., et al., Immunity 15 (2001) 445-455; Stüber, E., and Strober, W., J. Exp. Med. 183 (1996) 979-989; Stüber, E., et al., Gastroenterology 115 (1998) 1205-1215; Takahashi, Y., et al., J. Virol. 75 (2001) 6748-6757; Takasawa, N., et al., Jpn. J. Cancer Res. 92 (2001) 377-382; Taylor, L., and Schwarz, H., J. Immunol. Meth. 255 (2001) 67-72; Weinberg, A. D., et al., Nature Medicine 2 (1996) 183-189; Weinberg, A. D., et al., Semin. Immunol. 10 (1998) 471-480; Weinberg, A. D., Trends Immunol. 23 (2002) 102-109; Wu, T., et al., Transplant. Proc. 33 (2001) 217-218; Higgins, L. M., et al., J. Immunol. 162 (1999) 486-493; and Yoshioka, T., et al., Eur. J. Immunol. 30 (2000) 2815-2823. Human OX40L is the ligand for human OX40 (CD134) which is transiently expressed on activated CD4+ T cells. Engagement of OX40 by its ligand leads to a costimulatory signal for T cell activation. OX40/OX40L interaction is described to create a bidirectional signal (Matsumura, Y., et al., J. Immunol. 163 (1999) 3007-3011; Kotani, A., et al., Immunol. Lett. 84 (2002) 1-7).

Further OX40/Ox40L interaction mediate adhesion of activated T-cell to endothelial cells in inflammatory tissues. As OX40L is only transiently expressed on activated B cells, DC and endothelial cells, antibodies to OX40L should selectively block T cell activation and endothelial cell adhesion during an inflammatory response but leave unactivated, peripheral T cells unaffected. Yoshioka, A., et al. (Eur. J. Immunol. 30 (2000) 2815-2823) demonstrated the therapeutic potential of a neutralizing anti-mOX40L mAb in a mouse model for rheumatoid arthritis. Administration of it dramatically ameliorated the disease severity. This antibody showed similar activities in other related disease models, e.g. inflammatory skin disease, experimental autoimmune disease (EAE), GVHD, urine inflammatory bowel disease (Yoshioka, A., et al., Eur. J. Immunol. 30 (1999) 2815-2823; Salek-Ardakani, S., et al., J. Exp. Med. 198 (2003) 315-324; Burgess, J. K., et al., J. Allergy Clin. Immunol. 113 (2004) 683-689; Hoshino, A., et al., Eur. J. Immunol. 33 (2003) 861-869; Arestides, R. S., et al., Eur. J. Immunol. 32 (2002) 2874-2880; Nohara, C., et al., J. Immunol. 166 (2001) 2108-2115; Weinberg, A. D., et al., J. Immunol. 162 (1999) 1818-1826; Higgins, L. M., et al., J. Immunol. 162 (1999) 486-493; Humphreys, I. R., et al., J. Exp. Med. 198 (2003) 1237-1242; Akiba, H., et al., J. Exp. Med. 191 (2000) 375-380; Ishii, N., et al., Eur. J. Immunol. 33 (2003) 2372-2381; Blazar, B. R., et al., Blood 101 (2003) 3741-3748; Tsukada, N., et al., Blood 95 (2000) 2434-2439; Akiba, H., et al., Biochem. Biophys. Res. Commun. 251 (1998) 131-136.

Antibodies against OX40L have been investigated for their anti-inflammatory effects in various disease models (Sugamura, K., et al., Nat. Rev. Immunol. 4 (2004) 420-431).

Tanaka, Y., et al, Int. J. Cancer 36, (1985) 549-555; Tozawa, H., et al., Int. J. Cancer 41 (1988) 231-238; and Miura, S., et al., Mol. Cell. Biol. 11 (1991) 1313-1325 describe mouse monoclonal antibodies named TARM-34 and TAG-34 that react with surface antigens of lines of human lymphocytes besring a human T-cell leukemia virus type-I (HTLV-I). TAG-34 antibody is commercially available from MBL International Corporation. TAG-34 binds also to OX40L.

Antibodies against OX40L are known from, e.g. WO 95/12673; WO 95/21915 and WO 99/15200. They have been investigated for their anti-inflammatory effects in various disease models. An example of a commercially available antibody binding to OX40L is TAG-34 which is commercially available from MBL International Corporation.

### SUMMARY OF THE INVENTION

In a first aspect, the invention relates to a pharmaceutical formulation comprising:

1 to 200 mg/mL of an antibody against OX40 ligand;

1 to 100 mM of a buffer;

0.001 to 1% of a surfactant;

(a) 10 to 500 mM of a stabilizer; or

(b) 10 to 500 mM of a stabilizer and 5 to 500 mM of a tonicity agent; or

(c) 5 to 500 mM of a tonicity agent;

at a pH in the range of from 4.0 to 7.0,

The formulation according to the invention can be in a liquid form, a lyophilized form or in a liquid form reconstituted from a lyophilized form.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The following definitions are set forth to illustrate and define the meaning and scope of the various terms used to describe the invention herein.

The term “buffer” as used herein denotes a pharmaceutically acceptable excipient, which stabilizes the pH of a pharmaceutical preparation. Suitable buffers are well known in the art and can be found in the literature. Preferred pharmaceutically acceptable buffers comprise but are not limited to histidine-buffers, citrate-buffers, succinate-buffers, acetate-buffers, phosphate-buffers, arginine-buffers or mixtures thereof. Still preferred buffers comprise L-histidine or mixtures of L-histidine and L-histidine hydrochloride with pH adjustment with an acid or a base known in the art. The abovementioned buffers are generally used in an amount of about 1 mM to about 100 mM, preferably of about 5 mM to about 50 mM and more preferably of about 10-20 mM. Independently from the buffer used, the pH can be adjusted at a value comprising about 4.0 to about 7.0 and preferably about 5.0 to about 6.5 and still preferably about 5.5 to about 6.5 with an acid or a base known in the art, e.g. hydrochloric acid, acetic acid, phosphoric acid, sulfuric acid and citric acid, sodium hydroxide and potassium hydroxide.

The term “surfactant” as used herein denotes a pharmaceutically acceptable excipient which is used to protect protein formulations against mechanical stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Preferred polyoxyethylenesorbitan-fatty acid esters are polysorbate 20, (sold under the trademark Tween 20™) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Preferred alkylphenolpolyoxyethylene esthers are sold under the tradename Triton-X. When polysorbate 20 (Tween 20™) and polysorbate 80 (Tween 80™) are used they are generally used in a concentration range of about 0.001 to about 1%, preferably of about 0.005 to about 0.2% and more preferably about 0.01% to about 0.1% w/v (weight/volume).

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit. Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose, most preferred is trehalose.

The term “amino acid” as used herein denotes a pharmaceutically acceptable organic molecule possessing an amino moiety located at α-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Amino acids are generally used in an amount of about 10 to 500 mM, preferably in an amount of about 10 to about 300 mM and more preferably in an amount of about 100 to about 300 mM.

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in an amount of about 10 mM to about 500 mM, preferably in an amount of about 10 to about 300 mM and more preferably in an amount of about 100 to about 300 mM.

A subgroup within the stabilizers are lyoprotectants. The term “lyoprotectant” denotes pharmaceutical acceptable excipients, which protect the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. Preferred lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine. Lyoprotectants are generally used in an amount of about 10 to 500 mM, preferably in an amount of about 10 to about 300 mM and more preferably in an amount of about 100 to about 300 mM.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, glutathione, cysteine, methionine, citric acid, EDTA. Antioxidants can be used in an amount of about 1 to about 100 mM, preferably in an amount of about 5 to about 50 mM and more preferably in an amount of about 5 to about 20 mM.

The term “tonicity agents” as used herein denotes pharmaceutically acceptable tonicity agents. Tonicity agents are used to modulate the tonicity of the formulation. The formulation can be hypotonic, isotonic or hypertonic. Isotonicity in general relates to the osmostic pressure relative of a solution usually relative to that of human blood serum. The formulation according to the invention can be hypotonic, isotonic or hypertonic but will preferably be isotonic. An isotonic formulation is liquid or liquid reconstituted from a solid form, e.g. from a lyophilised form and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids, sugars, in particular glucose. Tonicity agents are generally used in an amount of about 5 mM to about 500 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethylenglycols and salts. An example for a sugar which can at the same time be a stabilizer and a tonicity agent is trehalose.

The compositions described herein may also contain “adjuvants” such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The term “liquid” as used herein in connection with the formulation according to the invention denotes a formulation which is liquid at a temperature of at least about 2 to about 8° C. under atmospheric pressure.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. The lyophilisate has usually a residual moisture of about 0.1 to 5% (w/w) and is present as a powder or a physical stable cake. The lyophilizate is characterized by a fast dissolution after addition of a reconstitution medium.

The term “reconstituted formulation” as used herein in connection with the formulation according to the invention denotes a formulation which is lyophilized and re-dissolved by addition of reconstitution medium. The reconstitution medium comprise but is not limited to water for injection (WFI), bacteriostatic water for injection (BWFI), sodium chloride solutions (e.g. 0.9% (w/v) NaCl), glucose solutions (e.g. 5% glucose), surfactant, containing solutions (e.g. 0.01% polysorbate 20), a pH-buffered solution (eg. phosphate-buffered solutions).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

### Formulations

Exemplary antibodies against OX40L that may be used in the formulations of the invention are described in WO2006/029879 and include antibodies characterized in that said antibodies contain a Fc part from human origin, bind to OX40L and to denatured OX40L (in a Western Blot) in an antibody concentration of 100 ng. These antibodies bind to the same OX40L polypeptide epitope as the epitope to which the monoclonal antibody LC.001 binds. Such antibodies are e.g. LC.001, LC.033 and LC.060. These antibodies are preferably of human IgG1 type (wildtype) or do not bind human complement factor Clq and/or human Fcγ receptor on NK cells.

In one embodiment the invention provides a formulation comprising an antibody binding to OX40L characterized by comprising a variable light chain and a variable heavy chain, characterized in that the variable heavy chain comprises CDR1, CDR2 and CDR3 characterized in that CDR3 is selected from SEQ ID NOs: 33-38. It is especially preferred that CDR1 is selected from SEQ ID NOs: 21-25, CDR2 is selected from SEQ ID NOs: 26-32 and CDR3 is selected from SEQ ID NOs: 33-38

The antibody is preferably characterized by comprising a variable light chain and a variable heavy chain, characterized in that the variable light chain comprises CDR1, CDR2 and CDR3 characterized in that CDR3 is selected from SEQ ID NOs: 51-57. It is especially preferred that CDR1 is selected from SEQ ID NOs: 39-44, CDR2 is selected from SEQ ID NOs: 45-50. and CDR3 is selected from SEQ ID NOs: 51-57.

The antibody is preferably characterized by comprising a variable heavy chain and a variable light chain, characterized in that the variable heavy chain comprises CDR1, CDR2 and CDR3 characterized in that CDR3 of the heavy chain is selected from SEQ ID NOs: 33-38 and CDR3 of the light chain is selected from SEQ ID NOs: 51-57. It is especially preferred that the variable heavy chain comprises CDR1 selected from SEQ ID NOs: 21-25, CDR2 selected from SEQ ID NOs: 26-32 and CDR3 selected from SEQ ID NOs: 33-38 and the variable light chain comprises CDR1 selected from SEQ ID NOs: 39-44, CDR2 selected from SEQ ID NOs: 45-50 and CDR3 selected from SEQ ID NOs: 51-57.

All CDRs are selected independently from each other but as a matter of course in such a manner that the antibody binds to OX40L. Therefore CDRs of light and heavy chains of the same LC antibody can be combined or the light chain CDRs of LC.001 with the heavy chain CDRs of LC.001, LC.059 or LC.063. CDRs on each chain are separated by framework amino acids.

The antibody is preferably characterized in that the antibody comprises CDRs independently selected from the group consisting of
a) the light chain (V_{L}) variable CDRs of amino acid sequence SEQ ID NO:1 and the heavy chain (V_{H}) variable CDRs of SEQ ID NO:2;
b) the light chain variable CDRs of amino acid sequence SEQ ID NO:3 and the heavy chain variable CDRs of SEQ ID NO:4;
c) the light chain variable CDRs of amino acid sequence SEQ ID NO:5 and the heavy chain variable CDRs of SEQ ID NO:6;
d) the light chain variable CDRs of amino acid sequence SEQ ID NO:7 and the heavy chain variable CDRs of SEQ ID NO:8;
e) the light chain variable CDRs of amino acid sequence SEQ ID NO:9 and the heavy chain variable CDRs of SEQ ID NO:10;
f) the light chain variable CDRs of amino acid sequence SEQ ID NO:11 or 16 and the heavy chain variable CDRs of SEQ ID NO:12;
g) the light chain (V_{L}) variable domain defined by amino acid sequence SEQ ID NO:1 and the heavy chain (V_{H}) variable domain defined by SEQ ID NO:17;
h) the light chain variable domain defined by amino acid sequence SEQ ID NO:18 and the heavy chain variable domain defined by SEQ ID NO:19;
i) the light chain variable domain defined by amino acid sequence SEQ ID NO:1 and the heavy chain variable domain defined by SEQ ID NO:20;
   or an OX40L-binding fragment thereof.

The antibody is preferably characterized in that said antibody comprises a variable region independently selected from the group consisting of
a) the light chain (V_{L}) variable domain defined by amino acid sequence SEQ ID NO:1 and the heavy chain (V_{H}) variable domain defined by SEQ ID NO:2;
b) the light chain variable domain defined by amino acid sequence SEQ ID NO:3 and the heavy chain variable domain defined by SEQ ID NO:4;
c) the light chain variable domain defined by amino acid sequence SEQ ID NO:5 and the heavy chain variable domain defined by SEQ ID NO:6;
d) the light chain variable domain defined by amino acid sequence SEQ ID NO:7 and the heavy chain variable domain defined by SEQ ID NO:8;
e) the light chain variable domain defined by amino acid sequence SEQ ID NO:9 and the heavy chain variable domain defined by SEQ ID NO:10;
f) the light chain variable domain defined by amino acid sequence SEQ ID NO:11 or 16 and the heavy chain variable domain defined by SEQ ID NO:12;
g) the light chain (V_{L}) variable domain defined by amino acid sequence SEQ ID NO:1 and the heavy chain (V_{H}) variable domain defined by SEQ ID NO:17;
h) the light chain variable domain defined by amino acid sequence SEQ ID NO:18 and the heavy chain variable domain defined by SEQ ID NO:19;
i) the light chain variable domain defined by amino acid sequence SEQ ID NO:1 and the heavy chain variable domain defined by SEQ ID NO:20;
   or an OX40L-binding fragment thereof.

The antibody is preferably characterized in that the human light chain variable region comprises an amino acid sequence independently selected from the group consisting of SEQ ID NO: 1, 3, 5, 7, 9, 11, 16 and 18.

The antibody is preferably characterized in that the human heavy chain variable region comprises an amino acid sequence independently selected from the group consisting of SEQ ID NO: 2, 4, 6, 8, 10, 12, 17, 19 and 20.

The CDR regions of the heavy and light chains are shown in SEQ ID NO: 21-38 and 39-57.

The antibody is preferably characterized in that the antibody comprises the light chain variable domain defined by amino acid sequence SEQ ID NO:1 and the heavy chain variable domain defined by SEQ ID NO:2, 17 or 20.

The antibody is preferably characterized in that the human heavy chain constant region comprises an amino acid sequence independently selected from the group consisting of SEQ ID NO: 14 and 15 or the heavy chain constant region of SEQ ID NO:58.

The antibody is preferably characterized in that the antibody comprises a κ-light chain constant region of SEQ ID NO: 13 or the light chain constant region of SEQ ID NO:61, 65 or 69.

Preferably an antibody according to the invention is characterized of binding to OX40L and by being of human IgG1 class (wildtype) and comprises as γ heavy chain SEQ ID NO: 58, 62 or 66. Especially preferred is an antibody comprising as

a) γ heavy chain SEQ ID NO:58 and as kappa light chain SEQ ID NO:61,
b) γ heavy chain SEQ ID NO:62 and as kappa light chain SEQ ID NO:65 or
c) γ heavy chain SEQ ID NO:66 and as kappa light chain SEQ ID NO:69.

A further embodiment of the invention is a formulation comprising an antibody binding to OX40L, characterized in that it is produced by cell line hu-Mab<hOX40L>LC.001, hu-Mab<hOX40L>LC.005, hu-Mab<hOX40L>LC.010, hu-Mab<hOX40L>LC.019, hu-Mab<hOX40L>LC.029 or hu-Mab<hOX40L>LC.033, as described in WO2006/029879.

The antibody is preferably a chimeric, human or humanized antibody.

The antibody according to the invention is preferably characterized by binding to OX40L with a K_{D} value of less than 10⁻⁸ M (10⁻¹² to 10⁻⁸ M), more preferably by a K_{D} range of 10⁻¹² to 10⁻⁹ M in a BIAcore assay.

The antibody preferably inhibits the interaction of OX40L with OX40 in an ELISA assay using immobilized OX40L (preferably biotinylated OX40L immobilized on a streptavidine surface) at a coating concentration of 0.5 μg/ml with an IC50 value of no more than 4 nM. More preferred the IC50 value is in the range of 1 to 4 nM.

The antibody is preferably characterized in that non-binding of the antibody to complement factor Clq refers to an ELISA assay measurement wherein the maximal binding (Bmax) of the antibody at a concentration of 10 μg/ml to Clq is 30% or lower, preferably 20% or lower compared to Bmax of antibody LC.001.

Preferably the antibody does not bind to human FcγRI, FcγRIIA and/or FcγRIIIA. Especially preferred, the antibody does not bind to human Fcγ receptor on NK effector cells.

The antibody is preferably characterized in that non-binding of the antibody to the Fcγ receptor on NK cells refers to an assay wherein the maximal binding (Bmax) of the antibody at a concentration of 20 μg/ml to NK cells is 20% or lower, preferably 10% or lower compared to Bmax of antibody LC.001.

The antibody is preferably characterized in that it does not bind to FcγRI. This means that the antibody is characterized by an EC50 value which is five fold or more, preferably seven fold or more, such as eight fold or more compared to the EC50 value of LC.001, when measured in an assay testing binding of the antibody in a concentration ranging from 0.078 to 10 μg/ml to a B-cell lymphoma cell lacking FcγRIIA and FcγIIB, but expressing recombinant FcγRI.

The antibody is preferably characterized as being an IgG4 antibody or an IgG1 antibody comprising at least one amino acid mutation, preferably in the human Fc part, causing non-binding to complement factor Clq and/or non-binding to human Fcγ receptor on NK cells.

The antibody is preferably characterized in that it does not activate complement factor C3.

The antibody is preferably characterized by being of human subclass IgG4. In a further preferred embodiment of the invention, the formulation comprises an antibody which is characterized by being of any IgG class, preferably being IgG1 or IgG4, containing at least one mutation in E233, L234, L235, G236, D270, N297, E318, K320, K322, A327, A330, P331 and/or P329 (numbering according to EU index). Especially preferred are the IgG1 mutations PVA236, L234A/L235A and/or GLPSS331 as well as the IgG4 mutation L235E. It is further preferred that the antibody of IgG4 subclass contains the mutation S228P or the mutation S228P and L235E (Angal et al., Mol. Immunol. 30 (1993) 105-108).

The antibody, therefore, is preferably an antibody of human subclass IgG1, containing one or more mutation(s) from PVA236, GLPSS331 and/or L234A/L235A (numbering according to EU index).

Preferably the antibody is characterized by binding to OX40L, being of IgG1 class containing mutation L234A/L235A and comprises as γ heavy chain SEQ ID NO: 59, 63 or 67.

Especially preferred is an antibody comprising as

a) γ heavy chain SEQ ID NO:59 and as kappa light chain SEQ ID NO:61,
b) γ heavy chain SEQ ID NO:63 and as kappa light chain SEQ ID NO:65 or
c) γ heavy chain SEQ ID NO:67 and as kappa light chain SEQ ID NO:69.

Preferably the antibody characterized by being of IgG4 class containing mutation S228P comprises as γ heavy chain SEQ ID NO: 60, 64 or 68.

Especially preferred is an antibody comprising as

a) γ heavy chain SEQ ID NO:60 and as kappa light chain SEQ ID NO:61,
b) γ heavy chain SEQ ID NO:64 and as kappa light chain SEQ ID NO:65 or
c) γ heavy chain SEQ ID NO:68 and as kappa light chain SEQ ID NO:69.

The antibody according to the invention is preferably characterized in that it does not elicit complement-dependent cytotoxicity (CDC).

The antibody is preferably characterized in that it does not elicit antibody-dependent cellular cytotoxicity (ADCC).

The formulation of the invention, therefore, comprises anti-OX40L antibodies or single heavy or light chains characterized by their CDRs, variable regions, complete amino acid sequences or hybridomas and which comprise no Fc part or any type of Fc part, preferably human IgG1 Fc or human IgG4 Fc, either unmodified from human origin or modified by the above mentioned mutations.

The formulation of the invention, therefore, also comprises antibodies, preferably monoclonal antibodies, characterized in that said antibodies bind OX40L, contain a Fc part from human origin and do not bind human complement factor Clq and/or human Fcγ receptor on NK cells, by being of human IgG4 type or of human IgG1 or human IgG4 both modified by the above mentioned mutations.

The formulation of the invention, therefore, also comprises antibodies, preferably monoclonal antibodies, characterized in that said antibodies bind to OX40L and to denatured OX40L (in a Western Blot) in an antibody concentration of 100 ng. These antibodies bind to the same OX40L polypeptide epitope as the epitope to which the monoclonal antibody LC.001 binds. The antibodies comprise no Fc part or any type of Fc part, preferably human IgG1 or human IgG4, either wild-type or modified by the above mentioned mutations.

In one embodiment the present invention provides a formulation wherein the antibody is present in an amount in the range of from 10 to 150 mg/mL, preferably from 10 to 50 mg/mL.

The antagonistic monoclonal antibodies against OX40L may be produced by recombinant means, e.g. by those described in WO2006/029879. Such methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis) by standard techniques, including alkaline/SDS treatment, CsCl banding, column chromatography, agarose gel electrophoresis, and others well known in the art, e.g. as described in WO2006/029879.

In one embodiment, invention provides a pharmaceutical formulation comprising:

1 to 200 mg/mL of an antibody against OX40 ligand;

1 to 100 mM of a buffer;

0.001 to 1% of a surfactant;

(a) 10 to 500 mM of a stabilizer; or

(b) 10 to 500 mM of a stabilizer and 5 to 500 mM of a tonicity agent; or

(c) 5 to 500 mM of a tonicity agent;

at a pH in the range of from 4.0 to 7.0,

In another embodiment, invention provides a pharmaceutical formulation comprising:

1 to 50 mg/mL huMAb OX40L,

20 mM L-histidine HCl,

240 mM trehalose,

0.02% polysorbate 20,

at pH 6.0.

In another embodiment, invention provides a pharmaceutical formulation comprising:

1 to 50 mg/mL huMAb OX40L,

20 mM citrate buffer,

240 mM sucrose,

20 mM arginine

0.02% polysorbate 20,

at pH 5.5.

In another embodiment, invention provides a pharmaceutical formulation comprising:

1 to 50 mg/mL huMAb OX40L,

20 mM L-histidine HCl,

240 mM trehalose,

0.02% polysorbate 20,

at pH 6.0.

In another embodiment, invention provides a pharmaceutical formulation comprising:

1 to 50 mg/mL huMAb OX40L,

20 mM citrate buffer,

240 mM sucrose,

20 mM arginine

0.02% polysorbate 20,

at pH 5.5.

The formulations according to the invention are useful for prevention and/or treatment of inflammatory diseases in a mammal, preferably a patient suspected of having or suffering of such a disease. Such diseases include allergic reactions such as asthma. Other applications are the treatment of autoimmune diseases including rheumatoid arthritis.

Preferably the formulations of the present invention can be used for the treatment of severe persistent asthma in patients whose symptoms are not adequately controlled with inhaled corticosteroids. The patient population includes adults and adolescents (12 years of age and older) with inadequately controlled severe persistent asthma. The formulations will be delivered preferably subcutaneously once or twice a month. Main endpoint will be preferably decrease in acute exacerbations. Other endpoints include peak flow, daytime asthma symptoms, nocturnal awakenings, quality of life, emergency room visits, asthma free days, beta-2 agonist use, steroid reduction or tapering and effect on hyper-responsiveness.

It is further preferred to use the formulations according to the invention for monotherapy or in combination with methotrexate or other DMARDs (Disease Modifying Anti-Rheumatic Drugs) for the treatment of adults with moderate to severe active rheumatoid arthritis. It will be administered as subcutaneous injection every 2 or 4 weeks. It will be chronic therapy in patients who have failed one or more DMARDs. Endpoints will include reduction in signs and symptoms and the inhibition of progression of structural damage in adult patients with active rheumatoid arthritis. Prevention of disability, improvement in signs and symptoms measured by ACR criteria (ACR20 >60%, ACR50 >35%, ACR70 >15%; index from the American College of Rheumatology; www.rheumatology.com).

The invention further comprises the use of a formulation according to the invention for the manufacture of a medicament for asthma treatment.

A composition of the present invention can be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

The composition must be sterile and fluid to the extent that the composition is deliverable by syringe. In addition to water, the carrier can be an isotonic buffered saline solution, ethanol, polyol (e.g., glycerol, propylene glycol, and liquid polyetheylene glycol, and the like), and suitable mixtures thereof.

The formulation according to the invention can be administered by intravenous (i.v.), subcutaneous (s.c.) or any other parental administration means such as those known in the pharmaceutical art.

The formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found hereinafter.

### EXAMPLES

### Example 1

### Preparation of Liquid Formulations

Formulations of huMAb OX40L at a concentration of approx. 20 mg/mL were prepared by homogenization of solutions of huMAb OX40L in the production buffer (e.g. 20 mM histidine buffer at pH approx. 6.0 containing 240 mM trehalose and 0.02% (w/v) polysorbate 20, or 20 mM citrate buffer at pH 5.5 containing 240 mM sucrose, 20 mM arginin and 0.02% (w/v) polysorbate 20).

All formulations were sterile-filtered through 0.22 μm low protein binding filters and aseptically filled under nitrogen atmosphere into sterile 6 mL glass vials closed with ETFE (Copolymer of ethylene and tetrafluoroethylene)-coated rubber stoppers and alucrimp caps. The fill volume was approx. 2.4 mL. These formulations were stored at different climate conditions (5° C., 25° C. and 40° C.) for different intervals of time and stressed by shaking (1 week at a shaking frequency of 200 min⁻¹ at 5° C. or 25° C., respectively) and freeze-thaw stress methods. Samples were analyzed before and after applying the stress tests by 1) UV spectrophotometry, and 2) Size Exclusion Chromatography (SEC).

Size Exclusion Chromatography (SEC) was used to detect soluble high molecular weight species (aggregates) and low molecular weight hydrolysis products (LMW) in the formulations. Analysis was performed on a Water Alliance 2795 HPLC instrument equipped with a TSKgel G3000 SWXL column (7.8×300 mm). Intact monomer, aggregates and hydrolysis products were separated by an isocratic elution profile using 0.2M K₂HPO₄/0.25M KCL, pH 7.0 as mobile phase, and were detected at a wavelength of 280 nm. UV spectroscopy, used for determination of protein content, was performed on a Varian Cary Bio UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ɛ 〈 \frac{{cm}^{2}}{mg} 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient ε.

Formulations prepared according to Example 1 are shown in Table 1.

TABLE 1
Formulation A
Storage at 2-8° C.
20 mg/mL MAb OX40L,
20 mM L-histidine HCl,
240 mM trehalose,
0.02% polysorbate 20,
at pH 6.0

| | Protein | | | |
|---|---|---|---|---|
| | conc. | Size Exclusion - HPLC | | |
| Timepoint | (mg/mL) | HMW (%) | Monomer (%) | LMW (%) |
| Initial | 18.7 | 1.4 | 97.8 | 0.8 |
| 1 months | 18.1 | 1.4 | 97.9 | 0.7 |
| 2 months | 18.7 | 1.3 | 98.0 | 0.7 |
| 3 months | 19.1 | 1.4 | 97.8 | 0.8 |

Formulation A
Storage at 40° C.
20 mg/mL MAb OX40L,
20 mM L-histidine HCl,
240 mM trehalose,
0.02% polysorbate 20,
at pH 6.0

| | Protein | | | |
|---|---|---|---|---|
| | conc. | Size Exclusion - HPLC | | |
| Timepoint | (mg/mL) | HMW (%) | Monomer (%) | LMW (%) |
| Initial | 18.7 | 1.4 | 97.8 | 0.8 |
| 1 months | 18.4 | 0.8 | 98.0 | 1.1 |
| 2 months | 18.4 | 0.7 | 96.0 | 3.1 |
| 3 months | 19.1 | 0.8 | 92.4 | 6.7 |

Formulation B
Storage at 2-8° C.
20 mg/mL MAb OX40L,
20 mM citrate buffer,
240 mM sucrose
20 mM arginine
0.02% polysorbate 20,
at pH 5.5

| | Protein | | | |
|---|---|---|---|---|
| | conc. | Size Exclusion - HPLC | | |
| Timepoint | (mg/mL) | HMW (%) | Monomer (%) | LMW (%) |
| Initial | 20.7 | 1.6 | 97.6 | 0.8 |
| 1 months | 19.9 | 1.5 | 97.7 | 0.8 |
| 2 months | 20.5 | 1.4 | 97.8 | 0.7 |

Formulation B
Storage at 40° C.
20 mg/mL MAb OX40L,
20 mM citrate buffer,
240 mM sucrose
20 mM arginine
0.02% polysorbate 20,
at pH 5.5

| | Protein | | | |
|---|---|---|---|---|
| | conc. | Size Exclusion - HPLC | | |
| Timepoint | (mg/mL) | HMW (%) | Monomer (%) | LMW (%) |
| Initial | 20.7 | 1.6 | 97.6 | 0.8 |
| 1 months | 19.4 | 0.7 | 92.7 | 6.6 |
| 2 months | 20.4 | 0.7 | 87.6 | 11.7 |

### Example 2

### Preparation of Lyophilized Formulations and Liquid Formulations Reconstituted from Lyophilized Formulations

Solutions of approx. 20 mg/ml MAB OX40 were prepared as described in Example 1 and lyophilized using the freeze-drying cycle reported in Table 2.

TABLE 2
Freeze-drying Cycle type I

| | | | | Vacuum |
|---|---|---|---|---|
| | Shelf | Ramp | Hold | Set |
| | temperature | Rate | time | point |
| Step | (° C.) | (° C./min) | (min) | (μbar) |
| Pre-cooling | 5° C. | 0.0 | 60 | — |
| Freezing | −40° C. | 1.0 | 150 | — |
| Primary Drying | −25° C. | 0.5 | 3660 | 80 |
| Secondary Drying | +25° C. | 0.2 | 300 | 80 |

The product was first cooled from room temperature to approx 5° C. (pre-cooling), followed by a freezing step at −40° C. with a plate cooling rate of approx. 1° C./min, followed by a holding step at −40° C. for about 2 hours. The first drying step was performed at a plate temperature of approx. −25° C. and a chamber pressure of approx. 80 μbar for about 62 hours. Subsequently, the second drying step started with a temperature ramp of 0.2° C./min from −25° C. to 25° C., followed by a holding step at 25° C. for at least 5 hours at a chamber pressure of approx. 80 μbar.

Lyophilization was carried out in an Usifroid SMH-90 LN2 freeze-dryer (Usifroid, Maurepas, France). All lyophilized cakes had a residual water content of about 0.1 to 2.0% as determined by the Karl-Fischer method. The freeze-dried samples were incubated at different temperatures for different intervals of time.

The lyophilized formulations were reconstituted to a final volume of 5.3 mL with water for injection (WFI) yielding an isotonic formulation with an antibody concentration of approx. 20 mg/mL. The reconstitution time of the freeze-dried cakes was below 4 min. Analysis of the reconstituted samples was either performed immediately after reconstitution, or after a 24 hour incubation period of the reconstituted liquid sample at 25° C.

The samples were analyzed by 1) UV spectrophotometry and 2) Size Exclusion Chromatography (SEC).

Formulations prepared according to Example 2 are shown in Table 3.

TABLE 3
Formulation C
Storage at 2-8° C.
20 mg/mL MAb OX40L,
20 mM L-histidine HCl,
240 mM trehalose,
0.02% polysorbate 20,
at pH 6.0

| | Protein | | | |
|---|---|---|---|---|
| | conc. | Size Exclusion - HPLC | | |
| Timepoint | (mg/mL) | HMW (%) | Monomer (%) | LMW (%) |
| Initial | 20.9 | 0.4 | 98.8 | 0.8 |
| 1 months | 20.6 | 0.4 | 98.7 | 0.8 |
| 3 months | 20.6 | 0.4 | 98.8 | 0.8 |
| 6 month | 20.4 | 0.4 | 98.9 | 0.7 |
| 9 month | 20.6 | 0.4 | 98.8 | 0.8 |
| 12 month | 20.7 | 0.5 | 98.7 | 0.8 |

Formulation C
Storage at 40° C.
20 mg/mL MAb OX40L,
20 mM L-histidine HCl,
240 mM trehalose,
0.02% polysorbate 20,
at pH 6.0

| | Protein | | | |
|---|---|---|---|---|
| | conc. | Size Exclusion - HPLC | | |
| Timepoint | (mg/mL) | HMW (%) | Monomer (%) | LMW (%) |
| Initial | 20.9 | 0.4 | 98.8 | 0.8 |
| 1 months | 20.7 | 0.5 | 98.7 | 0.8 |
| 3 months | 20.7 | 0.5 | 98.6 | 0.8 |
| 6 month | 20.5 | 0.6 | 98.7 | 0.7 |
| 9 month | 20.5 | 0.6 | 98.6 | 0.8 |

Formulation D
Storage at 2-8° C.
20 mg/mL MAb OX40L,
20 mM citrate buffer,
240 mM sucrose
20 mM arginine
0.02% polysorbate 20,
at pH 5.5

| | Protein | | | |
|---|---|---|---|---|
| | conc. | Size Exclusion - HPLC | | |
| Timepoint | (mg/mL) | HMW (%) | Monomer (%) | LMW (%) |
| Initial | 21.0 | 1.6 | 97.6 | 0.8 |
| 1 months | 21.3 | 1.5 | 97.7 | 0.8 |
| 2 months | 21.3 | 1.5 | 97.8 | 0.7 |

Formulation D
Storage at 40° C.
20 mg/mL MAb OX40L,
20 mM citrate buffer,
240 mM sucrose
20 mM arginine
0.02% polysorbate 20,
at pH 5.5

| | Protein | | | |
|---|---|---|---|---|
| | conc. | Size Exclusion - HPLC | | |
| Timepoint | (mg/mL) | HMW (%) | Monomer (%) | LMW (%) |
| Initial | 21.0 | 1.6 | 97.6 | 0.8 |
| 1 months | 21.4 | 1.6 | 97.6 | 0.8 |
| 2 months | 20.9 | 1.6 | 97.5 | 0.7 |

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A pharmaceutical formulation comprising:
1 to 200 mg/mL of an antibody against OX40 ligand;
1 to 100 mM of a buffer;
0.001 to 1% of a surfactant;
(a) 10 to 500 mM of a stabilizer; or
(b) 10 to 500 mM of a stabilizer and 5 to 500 mM of a tonicity agent; or
(c) 5 to 500 mM of a tonicity agent;
at a pH in the range of from 4.0 to 7.0,

2. The formulation according to claim 1 wherein the antibody is characterized in that said antibody binds OX40L, contains a Fc part derived from human origin and does not bind complement factor Clq.

3. The formulation according to claim 1, wherein the antibody concentration is in the range of 10 mg/ml to 50 mg/mL.

4. The formulation according to claim 1 wherein the stabilizer is present in the formulation in an amount of 100 mM to 300 mM.

5. The formulation according to claim 1 wherein the surfactant is present in the formulation in an amount of 0.005 to 0.2% w/v.

6. The formulation according to claim 1 wherein the buffer is present in the formulation in an amount in the range of 5 mM to 50 mM.

7. The formulation according to claim 1, which comprises a tonicity agent.

8. The formulation according to claim 1, wherein the tonicity agent is present in the formulation in an amount in the range of 50 mM to 300 mM.

9. The liquid formulation of claim 1 which comprises:
1 to 50 mg/mL huMAb OX40L,
20 mM L-histidine HCl,
240 mM trehalose,
0.02% polysorbate 20,
at pH 6.0.

10. The liquid formulation of claim 1 which comprises:
1 to 50 mg/mL huMAb OX40L,
20 mM citrate buffer,
240 mM sucrose,
20 mM arginine
0.02% polysorbate 20,
at pH 5.5.

11. The lyophilized formulation according to claim 1 comprising:
1 to 50 mg/mL huMAb OX40L,
20 mM L-histidine HCl,
240 mM trehalose,
0.02% polysorbate 20,
at pH 6.0.

12. The liquid formulation of claim 1 which comprises:
1 to 50 mg/mL huMAb OX40L,
20 mM citrate buffer,
240 mM sucrose,
20 mM arginine
0.02% polysorbate 20,
at pH 5.5.

13. A method of treating asthma or allergy, the method comprising administering to a patient in need thereof a formulation of claim 1.
